(19) United States
(12) Patent Application Publication
(10) Pub. No.: US 2017/0266039 A1
(43) Pub. Date: Sep. 21, 2017

(54) HEAT STORAGE-TYPE FOOT FOMENTING DEVICE FOR PROMOTING ENERGY CIRCULATION

(71) Applicant: Seong Jin SONG, Daegu (KR)

(72) Inventor: Seong Jin SONG, Daegu (KR)

(21) Appl. No.: 15/313,968

(22) PCT Filed: Jun. 25, 2015
(86) PCT No.: PCT/KR2015/006461

§ 371 Date: Apr. 7, 2017

(30) Foreign Application Priority Data
Jun. 30, 2014 (KR) 10-2014-0080977

(51) Int. Cl. A61F 7/02

(52) U.S. Cl. CPC A61F 7/02; A61F 2007/0045

(57) ABSTRACT

Provided is a foot fomentation apparatus for feet of a user which includes: a soleplate which supports the soles; a cover which is combined with the soleplate from above for wrapping up feet; a warming and heating device which has a warming and heating structure in the soleplate and the cover as well or in the soleplate only; and a control unit which is installed in order to store heat energy in the warming and heating structure of the warming and heating device according to pre-established conditions. The present disclosure provides the apparatus of a heat-accumulative type, wherein no influence of electromagnetic waves exists and tension while relaxation coexist, in order to exuberantly promote energy and blood circulation to reduce fatigue and help preventing serious diseases by continuously inducing energy and blood circulation action in a human body, converting a half-healthy constitution into a healthy constitution, considerably improving work concentration with proper stimuli which incur no distraction when a user performs another work in experiencing the fomentation, pleasantly maintaining biorhythms and exuberantly maintaining streams of energy and blood along the twelve meridian points.

### BACKGROUND OF THE DISCLOSURE

1. Field of the Disclosure

The present disclosure relates to a heat-accumulative foot fomentation apparatus more particularly a heat-accumulative foot fomentation apparatus for promoting energy and blood circulation which reduces fatigue and improves work concentration by continuously inducing energy and blood circulation action in a human body.

2. Description of the Background Art

A human body should be kept warm in feet and the lower abdomen and, at the same time, cool at the head and above the shoulders as far as possible, which is the principle of head-cool-feet-warm or water-upward-fire-downward. Modern people show, however, health conditions opposite to the principle due to reasons caused by an excess of stress, nutritional unbalance, excessive eating, lack of exercise, and others. When such conditions continue, they can often develop into serious diseases such as cerebral infarction and myocardinal infarction or have trouble with modern lifestyle diseases.

A person who has a cold constitution should be treated with thermotherapeutic fomentation. For that reason, sleeping socks have been developed for patients suffering from cold hypersensitivity in their feet. However, it is important for such patients to consistently stimulate their feet with intense heat and give their feet thermotherapeutic fomentation, not for simply keeping their foot temperature by means of, for example, wearing such sleeping socks.

Speaking of oriental medicine, it is thought that a thing that has life has meridian points. In short, energy circulates through meridian points while the driving force to circulate blood originates from the energy. A human body has twelve meridian points, which control the five viscera and the six entrails. Six of the twelve meridian points are directly connected with while the remaining six are directly connected with hands. Since the six meridian points connected to are interconnected directly or indirectly to the remaining six, there are reflex points which correspond to all human organs in. Therefore, are referred to as a “microcosm of a human body”.

The twelve meridian points control streams of energy and blood in all organs and in every corner of a human body. When are consistently given fomentation with heat, energy and blood circulation will be exuberantly generated all around the human body because the twelve meridian points are stimulated in a conformable way. Therefore, it is very important to consistently stimulate feet. On the contrary, massage or acupressure should be practiced intermittently, not being able to consistently stimulate feet, while feet themselves can hardly tolerate consistent massage or acupressure.

Meanwhile, there are various products for stimulating or warming feet on the market. Among them, foot baths with hot water and foot massagers or foot acupressure apparatuses are widely known for keeping the thermotherapeutic condition in general.

However, foot baths are inconvenient in terms of use and management because hot water is poured into the foot baths and restricted in terms of location of use. As for a foot massager or the foot acupressure apparatuse, it is not easy for the user to concentrate on another work because the user is distracted due to mechanical stimuli. Especially, the latter has a short service life because it has an actuating unit which can easily and unexpectedly bring about an adverse effect with excessive stimuli.

Korean Unexamined Utility Model Publication No. 2012-0006750 (Prior Literature 1), Korean Examined Utility Model Publication No. 0215575 (Prior Literature 2) and others are known as examples of the prior arts related to foot stimulation.

Prior Literature 1 includes a large foot casing which accepts and warms feet from above the upper ankles and a small foot casing which accepts feet from beneath the ankles. It has a heat radiating fabric between the casing include an external skin made of one of artificial leather, polyester, cotton and microfiber, and a finishing rug which shows a good sense of touch and good thermal insulation, all of them are sewn together into one piece, wherein heat generated by the heat radiating fabric is introduced to the foot casing for warming feet and fomentation effects are expected when high-temperature heat is generated by raising the temperature by a temperature controller.

Prior Literature 2 suggests a configuration including a foot cover made of cloth, a fomentation pack which is inserted in the foot cover, formed into a bendable plate and includes inside heating lines and heat maintaining members the fomentation pack, and an external skin for tightly sealing and protecting the fomentation pack, wherein electric power is supplied from one side of the fomentation pack. This configuration further has a shoe-shaped protecting cover for maintaining the overall outward form so that a user can use the configuration in an easy and convenient way when wearing the shoes.

However, according to the Prior Literatures, they employ an electric heater for simple configurations and are influenced by electromagnetic wave, which considerably reduces the fomentation effects and involves safety accidents such as an electric shock.

### SUMMARY OF THE DISCLOSURE

To resolve the problems, the present disclosure provides the heat-accumulative foot fomentation apparatus for promoting energy and blood circulation which is of a heat-accumulative type for controlling heat intensity and tension level for pleasantly maintaining biorhythms and exuberantly maintaining streams of energy and blood along the twelve meridian points.

To achieve this objective, the present disclosure provides the foot fomentation apparatus which includes:

a soleplate which supports the soles;

a cover which is combined with the soleplate from above for wrapping up feet;

a warming and heating device which has a warming and heating structure in at least one of the soleplate and the cover; and

a control unit which is installed in order to store heat energy in the warming and heating structure of the warming and heating device according to a pre-established condition.

According an aspect of the present disclosure, the soleplate has continuously inducing energy and blood circulation in a human body, converting a half-healthy constitution into a healthy constitution, and

considerably improving work concentration with proper stimuli which incur no distraction when a user performs another work in experiencing the fomentation, and pleasantly maintaining biorhythms and exuberantly maintaining streams of energy and blood along the twelve meridian points.

According to an aspect of the present disclosure, a support on the bottom surface of which the tilt angle can be adjusted and an auxiliary part on the top side which supports the arch of the sole.

According to an aspect of the present disclosure, the soleplate has an external skin member which contains a substance which radiates far infrared rays.

According to an aspect of the present disclosure, the cover has ventilation holes for ventilating the area where the toes are placed.

According to a modified example of the present disclosure, the cover is further furnished with at least one of ankle covers and cilia for increasing thermal insulation performance.

The present disclosure provides the foot fomentation apparatus of a heat-accumulative type, wherein no influence of electromagnetic waves exists while tension and relaxation coexist, in order to exuberantly promote energy and blood circulation in order to reduce fatigue and help preventing serious diseases by

### BRIEF DESCRIPTION OF THE SEVERAL VIEWS OF THE DRAWING

The above and other features and advantages of the present disclosure will be more clearly understood from the following detailed description taken in conjunction with the accompanying drawing, in which:

FIG. 1a is an oblique view of the front surface of the foot fomentation apparatus according to the present disclosure;

FIG. 1b is an oblique view of the rear surface of the foot fomentation apparatus according to the present disclosure;

FIG. 2 is a side view of the foot fomentation apparatus according to the present disclosure;

FIG. 3 is a bottom view of the fomentation apparatus according to the present disclosure;

FIG. 4a is a cross-sectional view at A-A′ and B-B′ in FIG. 1b;

FIG. 4b is a cross-sectional view at another A-A′ and B-B′ in FIG. 1b;

FIG. 5 is a structural view of a modified example according to the present disclosure;

FIG. 6 is a side view of another substructure of the fomentation apparatus according to the present disclosure;

FIG. 7a is a partly cut side view which illustrates a hinged substructure of the fomentation apparatus described in FIG. 1; and

FIG. 7b is a partly cut side view which illustrates the hinged substructure of the fomentation apparatus described in FIG. 6.

### DETAILED DESCRIPTION OF THE DISCLOSURE

Terms and words used in the present Specifications and Claims should not be limited to common or lexical meaning and be construed as meanings and concepts which accord with the technical thoughts of the present disclosure according to the principle that the inventor can appropriately define the concepts of the terms so as to describe their invention in the best way. Therefore, the embodiments described in the present Specification and the configurations illustrated in the drawing are simple examples of the present disclosure, not entirely representing the technical thoughts of the present disclosure, and should be construed as including various equivalents and conversions which can replace them at the time of applying the present Specifications.

The present disclosure will be described more fully hereinafter with reference to the accompanying drawings.

The present disclosure suggests the fomentation apparatus for a user's feet considering the fact that the Korean traditional room heating system of a heat-accumulative type is well harmonized with biorhythms. In other words, because a human body as a living thing is not fixed but changeable along such biorhythms, which are constantly changed and dynamic, the foot fomentation apparatus suitable to such streams is provided.

The foot fomentation apparatus has the soleplate (10). The soleplate (10) is made of a light resin or wood having impact resistance and heat resistance. Although the soleplate (10) is illustrated in relevant FIGs as it accepts both feet at the same time, any configuration for accepting only a single foot is not excluded. At least one part of the soleplate (10) can have sole-shaped concave grooves (not illustrated).

According to a detailed aspect of the present disclosure, the soleplate (10) has a fixed support ((12a)) and the hinged support ((12b)), of which the tilt angle can be adjusted at the bottom side, and the auxiliary part (18) which supports the arch of the sole.

The supports (12a), (12b) are installed at the front end of the bottom side where the toes are placed, of the soleplate (10) so that the user can put their feet on the apparatus or lay down their feet to insert feet into the apparatus in a comfortable posture. The tilt angle of the soleplate (10) is maintained between −25 and 25° and, desirably, between 3 and 8° (or between −3 and −8°). However, it is desirable to adjust the tilt angle considering the physical conditions of the user (ordinary people, patients and others) or configure the supports (12a), (12b) so that its height can be adjustable in the forward and reverse directions.

The auxiliary part (18) is installed on the top side of the soleplate (10) in the middle of the area where the arch of feet is placed in order to evenly distribute the force exerted on the sole of the user. The auxiliary part (10) is made of a resin having impact absorbance and wear resistance. It is desirable to make the position and the height of the auxiliary part (10) adjustable because the position of the sole arch varies depending on the physical conditions of the user. Materials for the auxiliary part (18) can be selected among resins, wood and others although ceramics, genuine barley stone, genuine jade and others which radiate far infrared rays are desirable.

According to a detailed aspect of the present disclosure, the soleplate (10) can further have the external skin member (15) which contains a substance which radiates far infrared rays. The external skin member (15) is better in terms of efficacy when it is made of a plate, including ceramic materials, which radiates far infrared rays. Nevertheless, warming and heating is delayed or weight increases when the plate is thick whereas the plate becomes vulnerable to destruction when it is thin. In this regard, the external skin member (15) can be made of a flexible material such as a fabric which is pulverized, impregnated with or accepts far infrared ray-radiating substance.

In addition, according to the present disclosure, the cover (20) is combined with the soleplate (10) from above to wrap up feet. The cover (20) wraps up the flank and top sides of feet to a sufficient extent when it is combined with the soleplate (10). The cover (20) can be attached to and detached from the soleplate (10) for washing and drying convenience.

According to a detailed aspect of the present disclosure, the cover (20) can additionally have the ventilation holes (22) for ventilating the area where the toes are placed. The ventilation holes (22) are formed in order to suppress or alleviate bacterial proliferation because a hot and humid environment can be formed inside the cover (20) when the foot fomentation is performed for a long period. The ventilation holes are not limited to a small size but include relatively large incised holes (not illustrated). When the number and quantity of the ventilation holes (22) are considerably increased, it is desirable to add an independent and detachable cover for opening and closing the ventilation holes (22). Specifically, it is advantageous to weave the heat maintaining members to a small size in the warming and heating devices (30), (30′) considering the cover (20) is vulnerable to deformation by an external force.

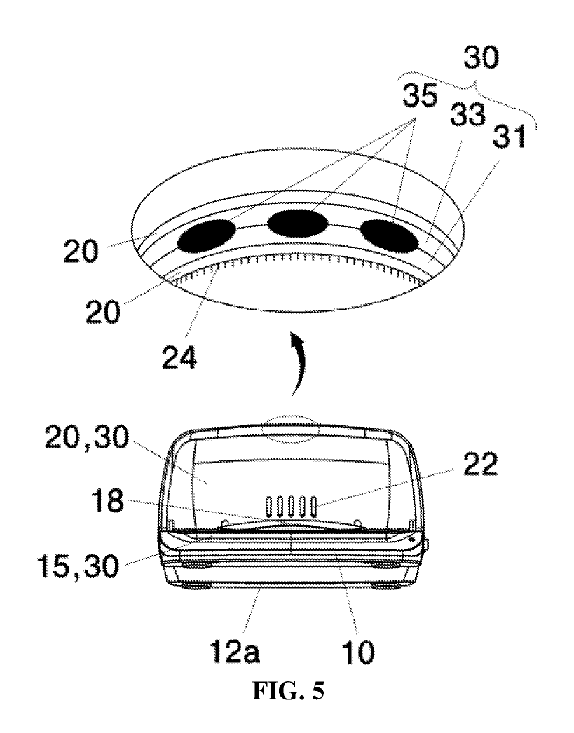

According to a modified example of the present disclosure (refer to FIG. 5), the cover (20) additionally has at least one of the ankle cover (26) and the cilia 24. The ankle cover is made of a flexible material and installed so that the ankle cover (26) extends from the end portion of the cover (20) toward the outside of the cover 20. When the ankle cover (26) is installed, an independent opening and closing device such as zip fasteners is added so that feet can come in and go out conveniently. The cilia (24) have a plurality of long hairs which are installed so that the hairs protrude from the end portion of the cover (20) toward its inside. Although the ankle cover (26) can be excluded when the cilia (24) are installed, the cilia (24) and the ankle cover (26) can be installed simultaneously.

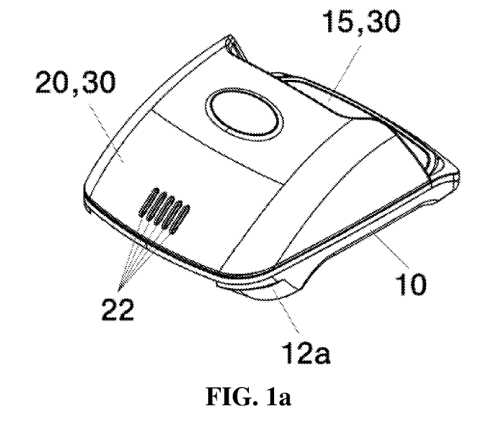

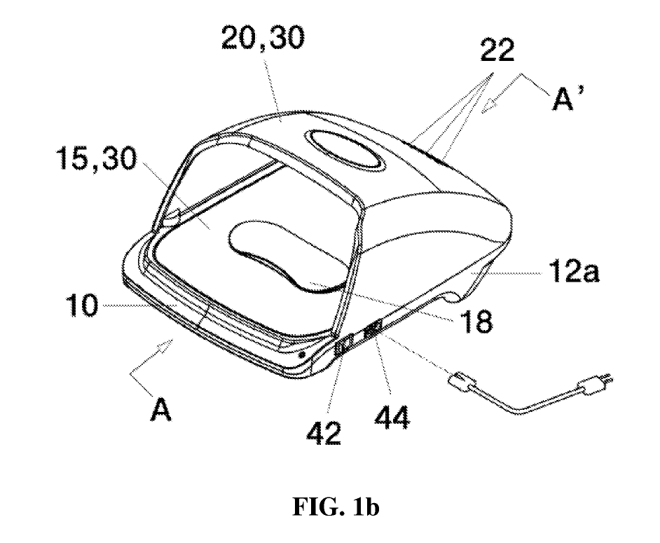

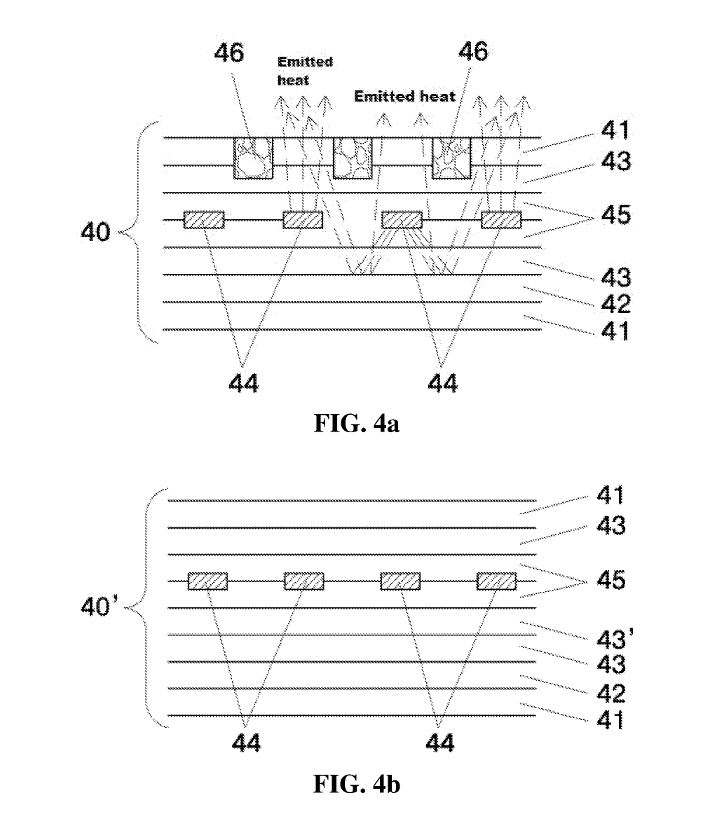

According to the present disclosure, the warming and heating device is inserted, when it is installed, in the soleplate (10) and the cover (20) or in the soleplate (10) only, wherein a heat radiating ceramic member (45) is used to transfer the heat to the human body. (The configuration at A-A′ in FIG. 1b corresponds to the warming and heating device denominated as (30) and (30′).) The warming and heating device (30), (30′) of FIG. 4 is installed inside the soleplate (10) or the cover (20) and there is no restriction of heat sources, although any electrical type is convenient to use. It is advantageous to wrap up the heat source with the heat maintaining members (43a), (43b), into which pulverized ceramics, vermiculite and silica stone are mixed. The heat maintaining members for the warming and heating device can be used after being filled in an independent resin bag or consolidated into the shape of a disc in a smaller size. According to an embodiment, wherein the warming and heating device is inserted into the soleplate (10) and the cover (20), as described in FIG. 4a, although the heat radiating ceramic member (45) is heated by means of electricity, electricity can be switched off at the moment using the apparatus commences and the fomentation is performed with accumulated heat as in the Korean traditional room heating system. Such heat accumulation can be continued for about 1 through 3 hours when protective pads (41a), ((41b)) are maintained at 40 to 80° C. Specifications (or capacity) of the heat radiating ceramic member can be established differently depending on its installation place, or the soleplate (10) and the cover (20), respectively.

According to a detailed aspect of the present disclosure, the warming and heating device (30) has the protective pads (41a), ((41b)) installed in the uppermost and lowermost portions of the heat radiating ceramic member (45), the heat maintaining member (43a) and a thermally insulating member (42) consecutively, and another one of the heat maintaining member (43b) above the heat radiating ceramic member (45) (Refer to FIG. 4a). It is required for the protective pads (41a), ((41b)) to have strength and wear resistance enough to endure any frequent use of the user. The protective pads (41a), ((41b)) can be made of aluminum alloy plates, copper plates, wood, high-strength resins or thick fabrics, to prevent the warming and heating device (30) from being damaged with external impact. The lower thermally insulating member (42) is to prevent the heat radiated from the heat radiating ceramic member (45) from being dissipated downward or into the ground, or conserve the heat, and can be made of nonflammable fiber, foam resins or foam ceramics. Although the structure of the protective pads (41a), ((41b)) can be omitted, it is advantageous to employ them all. Especially, it is desirable not to omit the upper thermally insulating member (43b) because it is efficient, from a functional perspective, to keep the heat radiated from the heat radiating ceramic member (45) to the extent possible.

Although it is advantageous, from a perspective of heat radiating structures, for the heat radiating ceramic member (45) to have a composition including 22 to 45% of kaolin, 15 to 35% of clay, 5 to 15% of agalmatolite, 5 to 15% of feldspar, 5 to 15% of silica, 2 to 10% of alumina, 2 to 10% of serpentine and others, such a component can be replaced by a substance having characteristics equivalent to those of the component, while one or two components can be omitted. Specifically, it is absolutely advantageous to include kaolin, clay, agalmatolite, feldspar, alumina or so. It is advantageous for a heating plate as a main part of the heat radiating ceramic member (45) to be made of ceramic materials to assume the shape of a stone plate. Coil- or pipe-shaped heating element (44) are inserted in the heat radiating ceramic member (45), wherein two (or a plurality) of the ceramic heating plates are laminated closely or attached to each other. The heat maintaining members (43a), (43b) have a composition, of which vermiculite and silica are mixed with each other with a rough ratio of 7 to 3, respectively, and form a porous light material having significantly many pores inside. The far infrared ray radiating substance (46) inserted in the uppermost protective pad ((41b)) and the upper heat maintaining member (43b) is made any one of barley stone, germanium, titanium dioxide, silver, tourmaline, zeolite, feldspar, serpentine, silica and others or a mixture of a few of them. The far infrared ray radiating substance (46) is not necessarily and additionally inserted. However, when it is added, it would be expected to have more efficacy of radiating far infrared rays, negative ions and others. The warming and heating device (30) in FIG. 4b is provided as an example of a configuration, for which part of the configuration of the warming and heating device (30) in FIG. 4a has been removed or added. That is to say, the thermally insulating member (42) or the heat maintaining members (43a), (43b) can be added or removed in order to maintain the heat of the heat radiating ceramic member (45) and emit the heat upward, not downward, concentrated or alleviated, (unlike the soleplate, the cover is to transfer the heat downward from the upper portion) when the heat radiating ceramic member (45) including the heating element (44) is a requisite element of the warming and heating device (30), (30′) according to the present disclosure. Although the warming and heating device (30) in FIG. 4b has a laminated structure, wherein two of the heat maintaining member (43a), 43a are overlapped above the thermally insulating member (42) in order for the heat radiating ceramic member (45) to store more of its heat in its inside and emit the heat upward for a long period, such a laminated structure is among examples subject to easy modification. The warming and heating device (30), (30′) can be inserted in the entire or a part of the internal surface of the soleplate (10) or the cover (20). When it is the soleplate (10), it is advantageous to install the warming and heating device across the entire internal surface as far as possible whereas, when it is the cover (20), it is advantageous to install the device in a part of the internal surface.

The control unit (50) according to the present disclosure maintains the warming and heating device (30), (30′) at pre-established temperature and displays operational states of the device. The control unit (50) includes a power source unit which converts AC to DC, a power cord inserting unit (52) (power socket unit) which is connected to the power source unit, a circuit board (51) which is installed inside the soleplate (10) or the cover (20), an operational switch (53) which is closely connected to the circuit board (51) and commences and stops operation, and a display unit (54) which displays operational states such as operational temperature, operational time and others. The control unit (50) can be installed on one side of the soleplate (10) or any other places. The circuit board 51 is a microcomputer circuit which has a control algorithm including a function of converting the domestic alternating current power to operational direct power. The operational switch (53) inputs an independent operation for the soleplate (10) and the cover 20, respectively, and a simultaneous operation for both of them. The display unit (54) displays the operational states, such as operational temperature and operational time, of the heat radiating ceramic member (45).

As for operation, after an operational mode is selected and input via the operational switch (53) of the control unit 50 is made, and after electric power is applied or supplied for about 3 to 10 minutes, the heat radiating ceramic member (45) is heated by means of the heating element (44). The temperature of the heat radiating ceramic member (45) exceeds about 100 to 250° C. after heating is over. While the heat radiating ceramic member (45) is maintained at about 200° C., the upper and lower heat maintaining members (43a), (43b) store heat at lower temperature, emitting the heat above the upper protective pad ((41b)), wherein the temperature of the contact surface of the upper protective pad (41b) reaches roughly 40 to 80° C. For 40 to 120 minutes, the contact surface is cooled slowly down below 40° C. When the contact surface is cooled to a certain degree, the electric power is automatically applied again whereas the user can manually turn on the operational switch (53). It is possible to adjust the temperature range of the heat radiating ceramic member (45) or heat transferred to the uppermost protective pad (41b). However, the user can suffer burns when heat at 80 to 90° C. or higher is transferred through the uppermost protective pad (41b) to the human body. Therefore, it is desirable to establish the temperature on an area which comes in contact with a human body to be about 40 to 80° C. When turning off the operational switch (53) and placing two or one of feet between the soleplate (10) and the cover (20), warmth of the heat radiating ceramic member (45) is constantly transferred to the human body for 30 to 300 minutes to perform the fomentation evenly onto the entire body effectively. In other words, the temperature of the warming and heating device (30), (30′) decreases from its peak value slowly to plot a declining curve, making warmth and fomentation effects continue for a few hours. With electric power applied for roughly 5 to 10 minutes, it is possible to perform fomentation by means of the accumulated heat with almost no influence of electromagnetic waves. During summer when ambient temperature is high or if a separate heat maintaining device such as blankets is used, the warmth transfer can be continued up to about 300 or 400 minutes. The temperature and time to heat the warming and heating device (30), (30′) can be established and updated with the control unit (50). When the user puts their feet in this way on the soleplate (10) of the fomentation apparatus according to the present disclosure in their work, the 12 meridian points are comfortably and naturally stimulated and the entire organs and tissues of the human body move silently but strenuously. That is the moment when crumpled body parts are disentangled, plugged parts are penetrated, cold energy ascends and warm energy descends.

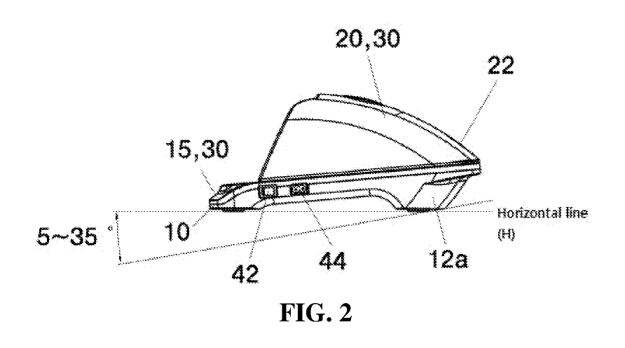

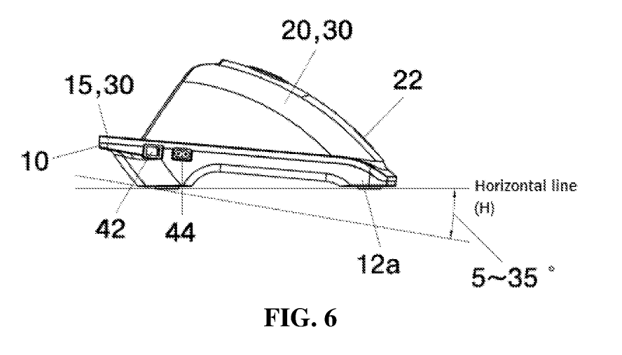

FIG. 6 describes the case where the tilt angle of the soleplate (10) is opposite to that of FIG. 2. In FIG. 2, the space, into which feet are initially inserted, is tilted down with reference to the horizontal line (H). Therefore, the toes rise upward and the heel drops down. On the contrary, in FIG. 6, the space, into which feet are initially inserted, is tilted up with reference to the horizontal line (H). Therefore, the toes drop down and the heel rises upward in the apparatus. Generally speaking from the perspective of health, the posture of FIG. 2 is desirable. However, quite a number of women have the opinion that they feel more comfortable when the apparatus is initially tilted as in FIG. 6 because they have adapted themselves to high heels. Therefore, the user can make the tilt angle of the soleplate (10) forward and reverse as in FIG. 2 and FIG. 6, respectively.

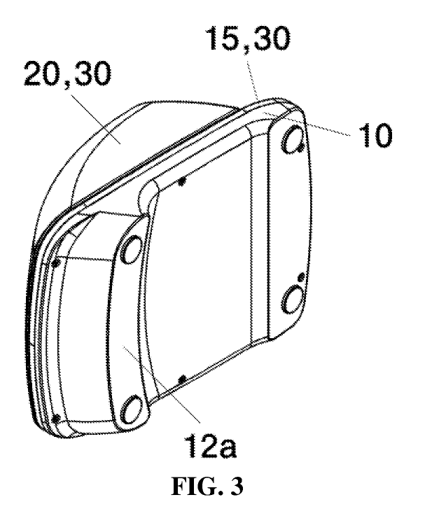

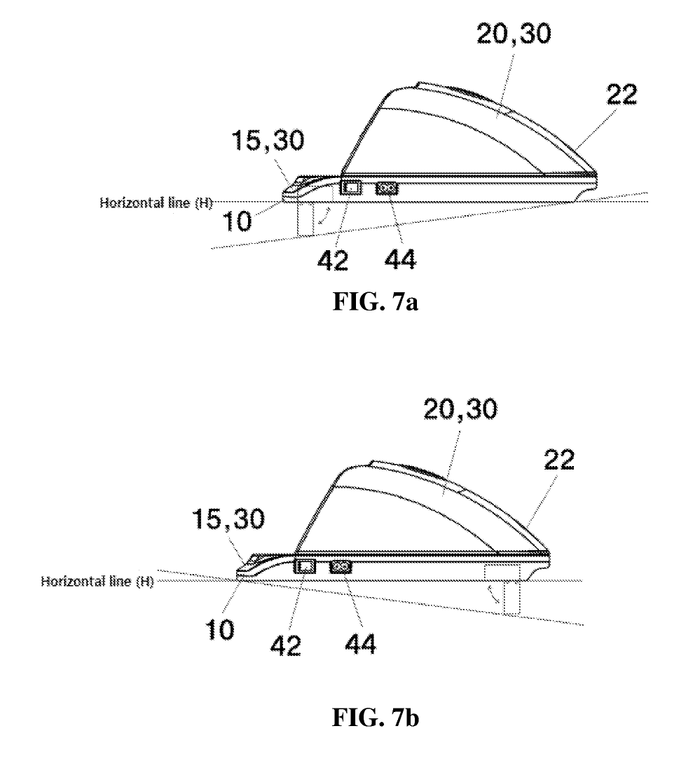

Although the foot fomentation apparatus according to the present disclosure can have the tilt angle as illustrated in FIG. 2 and FIG. 3, respectively, the hinged support (12b) can be independently installed in the lower portion of the soleplate (10) with the soleplate (10) maintained level. In this case, an internal slot (60), into which the hinged support (12b) can be inserted, and a hinge unit 61 can be formed at an appropriate place in the lower portion of the soleplate (10) while the hinged support (12b) can be drawn, if necessary, out of the internal slot (60) for using and then folded and inserted again into the internal slot (60). It is needless to say that the hinged support (12b) can be installed in the front surface and the rear surface of the soleplate (10) at the same time or in any one of them only, as illustrated in FIGS. 7A and 7B. Reference number (70) (non described) refers to the auxiliary support.

As described thus far, the present disclosure provides the foot fomentation apparatus of a heat-accumulative type, wherein no influence of electromagnetic waves exists while tension and relaxation coexist, in order to exuberantly promote energy and blood circulation to reduce fatigue and help preventing serious diseases by continuously inducing energy and blood circulation action in a human body, converting a half-healthy constitution into a healthy constitution, considerably improving work concentration with proper stimuli which incur no distraction when a user performs another work in experiencing the fomentation, pleasantly maintaining biorhythms and exuberantly maintaining streams of energy and blood along the twelve meridian points.

Those skilled in this technology will appreciate that the conceptions and specific embodiments disclosed in the foregoing description may be readily utilized as a basis for modifying or designing other embodiments for carrying out the same purposes of the present disclosure. Those skilled in this technology will also appreciate that such equivalent embodiments do not depart from the spirit and scope of the disclosure as set forth in the appended Claims.

### REFERENCE CHARACTERS

(10): Soleplate
(12a): Fixed support
(12b): Hinged support
(15): External skin member
(18): Auxiliary part
(20): Cover
(22): Ventilation holes
(24): Cilia
(26): Ankle cover
(30), (30′): Warming and heating device
(41a), ((41b)): Protective pads
(42): Thermally insulating member
(43a), (43b): Heat maintaining member
(45): Heat radiating ceramic member
(46): Far infrared ray radiating substance
(50): Control unit
(51): Circuit board
(52): Power cord inserting unit
(53): Operational switch
(54): Display unit
(60): Internal slot
(61): Hinge unit

What is claimed is:

1. A heat-accumulative foot fomentation apparatus for promoting energy and blood circulation as a fomentation apparatus for feet of a user, comprising:
a soleplate which supports the sole of the both feet simultaneously;
a cover which is combined with the soleplate from above for wrapping up feet;
a warming and heating device which has a warming and heating structure in the soleplate and the cover;
a control unit which is installed in order to store and control heat energy in the warming and heating structure of the warming and heating device according to pre-established conditions; and
an auxiliary part which supports the arch of feet on the top surface and is adjustable in terms of position and height,
wherein the warming and heating device comprises:
protective pads installed in the uppermost and lowermost portions of a heat radiating ceramic member;
a heat maintaining member and a thermally insulating member consecutively; and
another heat maintaining member above the heat radiating ceramic member.

2. The heat-accumulative foot fomentation apparatus for promoting energy and blood circulation of claim 1, wherein the soleplate comprises a support on the bottom surface of which the tilt angle the tilt angle can be adjusted.

3. The heat-accumulative foot fomentation apparatus for promoting energy and blood circulation of claim 1, wherein the soleplate comprises an external skin member which contains a substance which radiates far infrared rays and the cover further comprises ventilation holes for ventilating an area where the toes are placed.

4. The heat-accumulative foot fomentation apparatus for promoting energy and blood circulation of claim 1, wherein the cover further comprises at least one of ankle covers and cilia for increasing thermal insulation performance.
